SEQUENCE LISTING (3 sequences)

SEQ ID NO: 1 - DNA, 1986 bp, Zea mays
cagcagttggaaacctgccatatacatccatccttgcacatagctccgatccaggtccagctccaccaacccggccggaagcagcaagcctcttgtctcctgcacgtaactccagcctccagcgccacacgtacacctccgccggatcggaagaagatggtgtcgcctacggagccgcaggccgagacaacggtgttccgctcgacgcttccggacatcgccatcccggaccacctcccgctccacgactacgtcctggagcgcctggcggagcgccgcgaccgcgcgtgcctcatcgacggcgccacgggcgaaacgctcaccttcggcgacgtggaccgcctgtcacgacgcgtcgcggccgggatgcgcgcctgcctcggcgtccgcagcgggggcacggtgatgctgcttctcccaaactccgtggagttcgcactcgcgttcctcgcgtgctcccgcctcggcgccgccgccaccacggccaacccgctccacaccccgcccgagatcgccaagcaggcggcggcctccggcgccaccgtcgtcatcaccgagccggcgttcgtcggcaaggtgcgggggctcgccggcgtcgccgtcgtcgccacgggcgacggcgcagagggctgcgtctcgttctccgacctcgcttcctccgccgacgatggctcggcggcgctgcctgaggcggcggcggccatcgacgtggcgaacgacgtggtcgcgctgccgtactcgtccggcacgacggggctgcccaagggggtgatgctgtcgcaccgtgggctggtaaccagcgtggcgcagctcgtcgacggcgacaacccgaacctccacttccgggaggacgacgtcgtcctctgcgtgctgcccatgttccacgtgtactcgctgcactccatcctgctgtgcgggatgcgcgccggcgcggcgctcgtgatcatgaagcgcttcgacactctccgcatgttcgagctggtgaagaggcacggcatcacgatcgtgccgctcgtgctgcccatcgcggtggagatggtcaagagcgacgccatcgaccgccacgacctctcgtcggtgcgcatggtcatctcgggggccgcgcccatgggcaaggagctgcaggacctgctgcgcgccaagctccctcgcgccgtgctcggacagggttatgggatgacagaggcaggcccggtgctctcgatgtgcatggcgtttgccaaggagccgttaccggtgaagtccggtgcctgcggcacggtggtgaggaatgccgagctaaagatcatcgacccggagaccgggctgtccctccaccgcaaccagcccggggagatttgcatcaggggcaagcagttgatgaaagggtacctcaacaacccggaggcaacggcgaagaccatcgactcggaggggtggctgcacaccggagacattgggtacgtcgacgacggcgacgagatcttcatcgtcgaccggctcaaggagctcatcaagtacaaggggttccaagtcgctccggcggagctcgaggccatgctcatcgcccaccccagcatcgtcgacgccgccgtcgtccaaatgaaggatgactcctgcggcgagatcccggtggcgttcgtcgtggcgtccggcggctccgggatcaccgaggacgagatcaagcagtacgtggcgaaacaggtggtgttctacaagaggctgcacaagatcttcttcgtggaggccatccccaaggcgccatccggcaagattttaaggaaggatctgagagcaaagctggcgtctggattctccaacgggtcatcgtgttgatgcccctgagttctttctatgcgaaaacgaccccgattttagtaaatttttttatgctgaacaacctaaaaaagatatatatacagaaaacggtgtaaacaagaagcagtcaaccatgtacaagacatgttatctagataaatgaaagttcaggtttgagtaaaaaaaaaa

SEQ ID NO: 2 - DNA, 2006 bp, Zea mays
ccgcaccagccatcgtctccttccttccttcctatactaccatccaaccagctgcccagcgcaacgttacctgcccgacatccgacaagccagtccatccggcagcgagcaaaggtctgagatgggttccgtagacgcggcgatcgcggtgccggtgccggcggcggaggagaaggcggtggaggagaaggcggtggtgttccggtccaagctccccgacatcgagatcgacagcagcatggcgctgcacacctactgcttcgggaagatgggtgaggtggcggagcgggcgtgcctggtcgacgggctgacgggcgcgtcgtacacgtacgcggaggtggagtccctgtcccggcgcgccgcgtcggggctgcgcgccatgggggtgggcaagggcgacgtggtgatgagcctgctccgcaactgccccgagttcgccttcaccttcctgggcgccgcccgcctgggcgccgccaccaccacggccaacccgttctacaccccgcacgaggtgcaccgccaggcggaggcggccggcgcccggctcatcgtgaccgaggcctgcgccgtggagaaggtgcgggagttcgcggcggagcggggcatccccgtggtcaccgtcgacgggcgcttcgacggctgcgtggagttcgccgagctgatcgcggccgaggagctggaggccgacgccgacatccaccccgacgacgtcgtcgcgctgccctactcctccggcaccaccgggctgcccaagggcgtcatgctcacccaccgcagcctcatcaccagcgtcgcgcagcaggttgatggcgagaacccgaacctgtacttccgcaaggacgacgtggtgctgtgcctgctgccgctgttccacatctactcgctgaactcggtgctgctggccggcctgcgcgcgggctccaccatcgtgatcatgcgcaagttcgacctgggcgcgctggtggacctggtgcgcaggtacgtgatcaccatcgcgcccttcgtgccgcccatcgtggtggagatcgccaagagcccccgcgtgaccgccggcgacctcgcgtccatccgcatggtcatgtccggcgccgcgcccatgggcaaggagctccaggacgccttcatggccaagattcccaatgccgtgctcgggcaggggtacgggatgacggaggcaggccccgtgctggcgatgtgcctggccttcgccaaggagccgtacccggtcaagtccgggtcgtgcggcaccgtggtgcggaacgcggagctgaagatcgtcgaccccgacaccggcgccgccctcggccggaaccagcccggcgagatctgcatccgcggggagcagatcatgaaaggttacctgaacgaccccgagtcgacgaagaacaccatcgacaaggacggctggctgcacaccggagacatcggctacgtggacgacgacgacgagatcttcatcgtcgacaggctcaaggagatcatcaagtacaagggcttccaggtgccgccggcggagctggaggcgctcctcatcacgcacccggagatcaaggacgccgccgtcgtatcaatgaacgatgaccttgctggtgaaatcccggtcgccttcatcgtgcggaccgaaggttctcaagtcaccgaggatgagatcaagcaattcgtcgccaaggaggtggttttctacaagaagatccacaaggtcttcttcaccgaatccatccccaagaacccgtcgggcaagatcctgaggaaggacttgagagccaggctcgccgccggtgttcactgaggcccgtatgcagcttcttctcggacgggcaccacgctgctgcgaaaaaaaaggcgatgtaatggcggtaacactactattcatgaactggcaacagaagggacacgtattcctgtggaacacatgttgccagaaagaggttttagttgtcctgtttgttggccctgtgtgtaatgttcaataaaccgatatagacgtcgtctctaaaaaaaaaaaa

SEQ ID NO: 3 - DNA, 1360 bp, Zea mays
ctccaccgcggtggcggccgctctagaactagtggatcccccgggctgcaggaattcggcacgaggcgcggcgctggtccgcgccgtggcgtccggcggcggcggcggcggcgaggccgtgaacctgggcgagctcatcttcaacctgaccaagaacgtgacgttccgcgccgccttcggcacccgcgacggcgaggaccaggaggagttcatcgccatcctgcaggagttctcgaagctgttcggcgccttcaacgtcgtcgacttcctgccgtggctgagctggatggacctgcagggcatcaaccgccgcctccgcgccgcacgatccgcgctggaccggttcatcgacaagatcatcgacgagcacgtgaggcgggggaagaaccccgacgacgccgacgccgacatggtcgacgacatgctcgccttcttcgccgaggccaagccgcccaagaaggggcccgccgccgccgcggacggtgacgacctgcacaacaccctccggctcacgcgcgacaatatcaaggctatcatcatggacgtgatgtttggcgggacggagacggtggcgtcggcgatcgagtgggcgatggcggagatgatgcacagccccgacgacctgcgccggctgcagcaggagctcgccgacgtcgtaggcctggaccggaacgtgaacgagtcggacctggacaagctccccttcctcaagtgcgtcatcaaggagacgctccggctgcacccgccgatcccgctgctcctgcacgagaccgccggcgactgcgtcgtgggcggctactccgtgcccaggggctcccgcgtcatggtcaacgtgtgggccatcggccgccaccgcgcctcgtggaaggacgccgacgcgttccggccgtcgcgcttcacgcccgagggcgaggccgcggggctcgacttcaagggcggctgcttcgagttcctgcccttcggctccggccgccgctcgtgccccggcacggcgctgggcctgtacgcgctggagctcgccgtcgcccagctcgcgcacggcttcaactggtcgctgcccgacggcatgaagccctcggagctggacatgggcgacgtcttcggcctcaccgcgccgcgcgccacgaggctctacgccgtgcctacgccccggctcaactgccccttgtactgacgccatgcgcgggcgactgccattaccatcgtcccctcgggtgggtgtggggtacgggggtaggagtttggtgcctttctctgtcgtcttttttccctttaaaaaacatgcctggtcgatgttgtagggtgtgttgtagacagccattatcaattttttttattctcaaaaaaaaaaaaaaaaaaa

(19) United States
(12) Patent Application Publication
(10) Pub. No.: US 2003/0175732 A1
(43) Pub. Date: Sep. 18, 2003

(54) Identifying genes associated with a qtl corn digestibility locus

(76) Inventors: Pere Puigdomenech, Barcelona (ES); Pascual Perez, Chanonat (FR); Alain Murigneux, La Roche Blanche (FR); Jean-Pierre Martinant, Vertaizon (FR); Marie-Helene Tixier, Pont Du Chateau (FR); Joan Rigau, Barcelone (ES); Laura Civardi, Barcelone (ES); Tamara Maes, Barcelone (ES)

Correspondence Address: YOUNG & THOMPSON, 745 SOUTH 23RD STREET 2ND FLOOR, ARLINGTON, VA, 22202

(21) Appl. No.: 10/182,113

(22) PCT Filed: Jan. 29, 2001
(86) PCT No.: PCT/FR01/00272

(30) Foreign Application Priority Data
Jan. 28, 2000 (FR) 00/01152

(51) Int. Cl. C12Q 1/68; A01H 5/00

(52) U.S. Cl. 435/6; 800/320.1

(57) ABSTRACT

The invention concerns the identification and co-localisation between two QTL corn digestibility loci and genes involved in the synthesis of lignin, and the uses thereof in methods for producing digestible corn.

The present invention relates to the identification of genes associated with QTL corn digestibility and lignin quantity loci. Many phenotypic characteristics show a continuous variation in population, it being possible to quantify this variation. Among such characteristics, mention may be made, for example, of the number of branches, the earliness, the resistance to insects, the percentage of proteins in the grain, etc. It is accepted that this continuous distribution may be explained by assuming that several segregating loci influence the variation in the characteristic, to which effects of the environment may also contribute (de Vienne et al., 1998). Since the beginning of the 1980s, it has been customary to call these loci QTLs (for “Quantitative Trait Loci”). Differences in effects between wild-type alleles are thought to be responsible for the variation in quantitative characteristics (Helentjaris 1987, Beavis et al., 1991).

The authors of the present invention have been interested, among these quantitative characteristics, in corn digestibility. The nutrient quality of silage corns depends, in addition to the harvesting stage (evaluated by the percentage of solids, which must be between 30 and 35%), on the respective quantities of grains and stems and on the digestibility specific to each of these two components.

The digestibility value of the corn is difficult to evaluate. The digestibility of the stems depends mainly on the digestibility of the cell walls. The cell walls consist mainly of cellulose, of hemicellulose and of lignin. Lignin, unlike the other constituents, is not digested by polygastric animals and it is therefore considered to be limiting for the digestibility of forage. Correlations between quantity of lignin and digestibility have been published, but the results are contradictory. A negative correlation is generally observed when various stages of maturity are compared, whereas the correlation is less evident when a single stage of maturity is considered (Jung and Allen, 1995). This reflects the fact that the lignin only partly explains the overall variation in digestibility.

Initially, measurements of digestibility of silage corn are carried out by measuring the energy gain of ruminants fed using this silage. These in vivo tests are, however, expensive and require large quantities of plant material. Thus, indirect methods have therefore been developed: —measurement of digestibility in vitro in rumen liquor (Menke et al., 1979); —enzymatic measurement of digestibility in vitro (Aufrere and Demarquilly, 1989). Chemical measurements have also been used to quantify the wall constituents (Van Soest, 1963). Another method for evaluating digestibility involves the use of near-infrared spectrometry (Ronsin and Femenias, 1990). This technique makes it possible to predict various parameters of digestibility or of quantity of wall constituents.

The authors of the present invention have now succeeded in establishing a colocalization between corn digestibility QTL and certain genes, known to be involved in the synthetic pathways for lignin, it being possible to take advantage of this colocalization in particular for producing more digestible corns.

For this, they have used a method comprising:

i) determining, for several individuals of a segregating corn progeny, the genotype of a set of marker loci distributed all along the genome;

ii) measuring the digestibility for each of the individuals studied;

iii) establishing a correlation between the genotype of the marker loci and digestibility, allowing the digestibility QTLs to be localized;

iv) establishing a correlation between the digestibility QTL thus localized and at least one gene, said gene having been mapped beforehand, on the same individuals or other individuals of the same species, in the region of said marker loci correlated with said digestibility QTL.

The term “marker locus” is intended to mean a locus identified by a polymorphic marker which provides information regarding the genotype at this locus and, in part, at the neighboring loci due to genetic linkage. Among common markers, mention may be made of RFLP (restriction fragment length polymorphism), RAPD (random-amplified polymorphic DNA), AFLP (amplification fragment length polymorphism) or SSR (simple sequence repeats) markers.

The term “segregating progeny” is intended to mean a population of genetically heterogeneous plants of the same genetic derivation which are, consequently, related. Examples of segregating progeny include populations obtained by backcrossing, recombinant lines or F2 line populations.

The digestibility measurements as mentioned in step (ii) above were made by near-infrared spectrometry (NIRS). A device of the monochromator type (NIRS system 5000—FOSS) was used. The wall content and lignin content (percentage relative to the walls) and a measurement of enzymatic digestibility of the walls according to Aufrere (Aufrere and Demarquilly, 1989) were thus predicted.

The correlation between the genotype of the marker loci and digestibility, which allows the digestibility QTLs to be localized as mentioned in step (iii) above, may be established using biometric methods know to those skilled in the art (de Vienne, 1998). These biometric methods may be based on analyses marker-by-marker (Tanskley et al., 1982) or by taking two or more markers into account together (Landen and Botstein, 1989). By means of this method, described more precisely in the examples below, the authors of the present invention have succeeded in identifying several chromosomal regions as being QTL corn digestibility loci.

A subject of the present invention is more particularly a chromosomal region identified as being a QTL corn digestibility locus, characterized in that it is delimited by the markers UMC 67 and UMC 128 on corn chromosome 1, as indicated in FIG. 1.

These markers are known to those skilled in the art and are, for example, accessible via the “Maize Genome Database” of the University of Missouri (http://www.agron.missouri.edu).

This chromosomal region contributes to about 15% of the phenotypic variance of the lignin content and also to about 15% of the enzymatic digestibility.

The chromosomal region of interest is more particularly delimited by the markers UMC 67, or preferably UMC 58, at one end and by the marker UMC 128 at the other end.

By applying step (iv) of the method described above, the authors of the present invention have demonstrated that this chromosomal region, identified as being a QTL corn digestibility locus, comprises the 4CL2 gene encoding the 4-coumarate CoA ligase 2 enzyme, and the CCR gene encoding the cynnamoyl CoA reductase enzyme, localized between the markers UMC 58 and UMC 128.

These markers may in particular be used to prepare primers for amplification reactions of the PCR (polymerase chain reaction) type, applied to corn DNA, or to prepare probes for Southern hybridizations.

A subject of the invention is therefore a chromosomal region identified as being a QTL corn digestibility locus, characterized in that it is included between the markers UMC 67 and UMC 128 on corn chromosome 1, and in that it comprises the 4CL2 gene encoding the 4-coumarate CoA ligase 2 enzyme and the CCR gene encoding the cynnamoyl CoA reductase enzyme.

The attached sequence SEQ ID No. 1 corresponds to a full length cDNA encoding corn 4CL2.

U.S. patent application No. 866 791 describes, moreover, a recombinant DNA encoding the corn CCR enzyme, also described in the article by Civardi et al., 1998 and accessible on the GenBank database (accession No. Y13734).

The authors of the present invention have also demonstrated another chromosomal region, identified as being a QTL corn digestibility locus. This region is characterized in that it is delimited by the markers bnlg 1046 and bnlg 609 on corn chromosome 5, as indicated in FIG. 2.

These markers are known to those skilled in the art and are, for example, given by the “Maize Genome Database” of the University of Missouri.

The contribution of this chromosomal region relative to the lignin content is about 10% of the phenotypic variance.

The chromosomal region of interest is more particularly delimited by the marker UMC 27a at one end and by the marker bnl 7.71 at the other end.

By applying step (iv) of the method described above, the authors of the present invention have demonstrated that this chromosomal region, identified as being a QTL corn digestibility locus, comprises the 4CL1 gene encoding the 4-coumarate CoA ligase 1 enzyme, the CAD gene encoding the cinnamyl alcohol dehydrogenase enzyme and the F5H gene encoding the ferulate 5-hydroxylase enzyme, localized between the markers UMC 27a and bnl 7.71.

These markers may in particular be used to prepare primers for amplification reactions of the PCR (polymerase chain reaction) type, applied to corn DNA, or to prepare probes for hybridizations (Southern).

A subject of the invention is therefore a chromosomal region identified as being a QTL corn digestibility locus, characterized in that it is included between the markers bnlg 1046 and bnlg 609 on corn chromosome 5, and in that it comprises the 4CL1 gene encoding the 4-coumarate CoA ligase 1 enzyme, the CAD gene encoding the cinnamyl alcohol dehydrogenase enzyme and the F5H gene encoding a ferulate 5-hydroxylase.

The full length cDNA sequence encoding the 4CL1 enzyme is given in the attached sequence listing, SEQ ID No. 2.

The CAD enzyme and its gene are described in Civardi et al., 1998, and on the Genbank database (accession No. Y13733).

The attached sequence SEQ ID No. 3 corresponds to the cDNA encoding corn F5H.

The identification of digestibility QTLs and the demonstration of the markers and genes associated with these QTLs in accordance with the invention opens the way to a certain number of applications.

Nucleic acid probes or primers for PCR amplification may be constructed from all or part of the chromosomal regions described above or bordering them. They may in particular be probes obtained from the sequences encoding the CCR, 4CL2, CAD and/or 4CL1 and F5H enzymes.

Marker-assisted selection programs may be carried out using these probes as markers, so as to introgress chromosomal segments into corn varieties in order to increase the digestibility value of these varieties. A subject of the present invention is therefore the use of at least one nucleic acid probe or primer obtained from all or part of the chromosomal regions as defined above, or bordering them, for following the introgression of the corn digestibility QTL, in a marker-assisted corn selection program.

For example, these probes may be labeled according to conventional techniques known to those skilled in the art, for example with a radioactive isotope, and used to locate the digestibility QTLs in corn by virtue of the Southern technique for example. For this, at least one labeled probe is brought into contact with the genomic DNA of a corn, digested beforehand with restriction enzymes, under suitable hybridization conditions, and then the size of the restriction fragment which hybridizes with the probe is determined. Primers constructed from all or part of the chromosomal regions described above, or bordering these regions, may also be used to characterize these regions, according to conventional PCR-type techniques (using two flanking primers) or quantitative PCR techniques (using two flanking primers and a primer specific for the favorable allele), or alternatively by means of a technique which does not use PCR, such as that described and sold by Third Wave Technology (Madison, Wis. 53719.1256, USA), named Invader™.

A subject of the present invention is also a method for determining a correlation between the haplotype of the chromosomal regions identified as being a QTL corn digestibility locus and its digestibility value, comprising:

a) haplotyping all or part of the chromosomal region identified as being a QTL digestibility locus, this haplotyping step being carried out on individuals belonging to the progeny of corns on which the chromosomal regions as defined above have been identified as being a QTL corn digestibility locus; and/or

b) haplotyping all or part of the chromosomal region of the QTL digestibility locus as defined above, this haplotyping step being carried out on individuals which are not related to said individuals of step (a);

c) determining the digestibility value for all said individuals;

d) establishing a correlation between the digestibility value and the haplotype profiles obtained.

The haplotyping consists in investigating the assortments of alleles at the various markers of the QTL digestibility locus. The haplotying conventionally comprises revealing the haplotypes by sequencing all or part of the chromosomal regions defined above and typing or identifying the haplotype at the positions of sequences identified as polymorphic, by means of nucleotide probes or primers obtained from all or part of one of the chromosomal regions defined above, using conventional hybridization or amplification techniques.

The populations subjected to haplotyping may consist of individuals in the progeny of a backcross used to study QTL loci, for which the alleles favorable to high digestibility are known. They may also be unrelated individuals, i.e. individuals which have no direct common ancestors.

A correlation between the haplotype and the digestibility value is established by dividing the population up according to the haplotypes present. A correlation or association is observed if the means for the digestibility values vary significantly between the populations sorted according to molecular polymorphism.

Once these molecular polymorphisms linked to the digestibility characteristic have been identified, the individuals of unknown digestibility can be selected using the haplotyping of the QTL digestibility locus.

A subject of the present invention is therefore also a method for the predictive determination of the digestibility value of a corn, comprising:

a) the haplotyping of all or part of the chromosomal region identified as being a QTL digestibility locus;

b) the predictive determination of the digestibility value of said corn, by means of the previously established correlation.

Consequently, the selection of corn with high digestibility is then advantageous and may be carried out using a method comprising:

haplotyping all or part of the chromosomal region identified as being a QTL digestibility locus,

selecting, from these individuals, the plants which have a high predictive digestibility value determined using the method described above.

This method advantageously adds to the methods for selecting corn which has a high degree of digestibility, in which the following are carried out:

genotyping individuals using nucleic acid probes or primers obtained from all or part of a chromosomal region as defined above or bordering it;

selecting, from these individuals, the plants which comprise a high frequency of favorable alleles associated with digestibility.

All of these methods in fact make it possible to determine the digestibility value of any individuals, unrelated to the genetic material used for the QTL search. These selection methods more particularly allow the screening of corn representing genetic resources different from those conventionally used in selection and, via this, the selection of novel lines or populations with a high degree of digestibility. They also make it possible to obtain improved corn plants, with a high degree of digestibility, by the transfer (in particular in the form of introgression) of the alleles or haplotypes linked to a desired digestibility.

The invention also relates to a method for obtaining genetically transformed corn having a high digestibility, said method comprising:

(i) transforming a corn plant cell with at least two nucleotide sequences encoding enzymes which are different from one another, said sequences being chosen from a sequence of an allele favorable to high digestibility encoding CCR, a sequence of an allele favorable to high digestibility encoding 4CL2, a sequence of an allele favorable to high digestibility for 4CL1, a sequence of an allele favorable to high digestibility encoding CAD and a sequence of an allele favorable to high digestibility encoding F5H, said sequences being carried by one or more nucleic acids; and

(ii) regenerating a transgenic plant from the cell thus transformed.

The present invention extends, moreover, to a nucleic acid encoding the 4-coumarate CoA ligase 2 (4CL2) enzyme, comprising the nucleotide sequence SEQ ID No. 1.

The invention also covers a nucleic acid encoding the 4-coumarate CoA ligase (4CL1) enzyme, comprising the sequence SEQ ID No. 2.

A subject of the invention is also a nucleic acid encoding the ferulate 5-hydroxylase (F5H) enzyme, comprising the sequence SEQ ID No. 3.

The invention also relates to a vector comprising at least one of the nucleic acids mentioned above, in combination with functional sequences for the expression thereof. A method for obtaining a genetically modified corn, comprising transforming a corn plant cell with this nucleic acid or this vector and regenerating a transgenic plant from the cell thus transformed, is also included in the invention. Finally, the invention relates to a transgenic corn plant, or part of this plant, such as seed, leaf, cell or others, which can be obtained using said method.

The following examples and figures illustrate the invention without in any way limiting the scope thereof.

### LEGEND TO THE FIGURES

FIG. 1 represents the consensus map of the SSR markers of corn chromosome 1, accessible on the internet site (http://www.agron.missouri.edu/cgi-bin/sybgw mdb/mdb3/Map/145822).

FIG. 2 represents the consensus of the SSR markers of corn chromosome 5, accessible on the internet site (http://www.agron.missouri.edu/cgi-bin/sybgw mdb/mdb3/Map/145826).

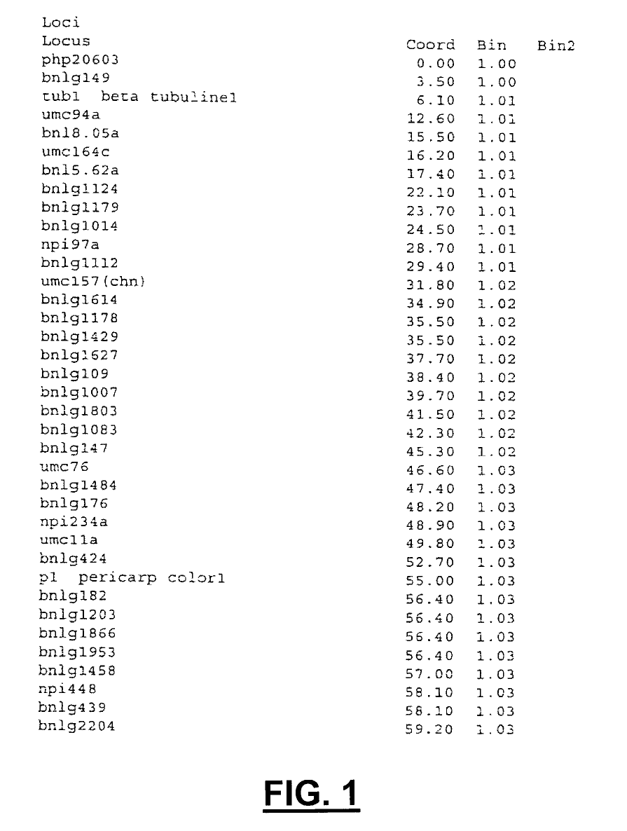

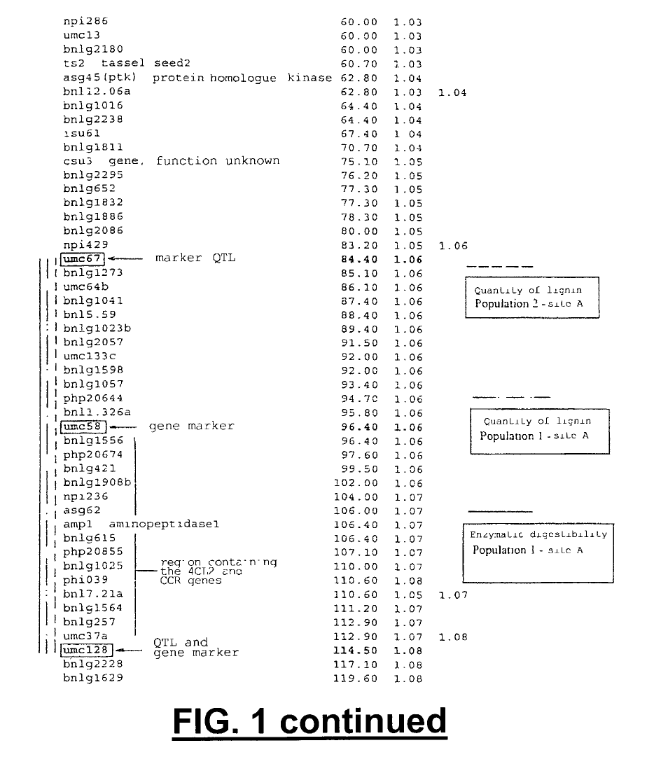

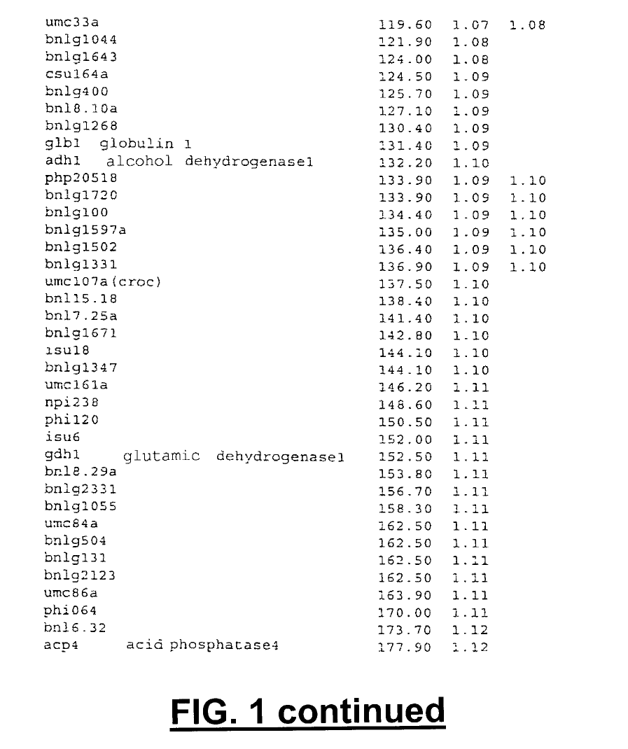

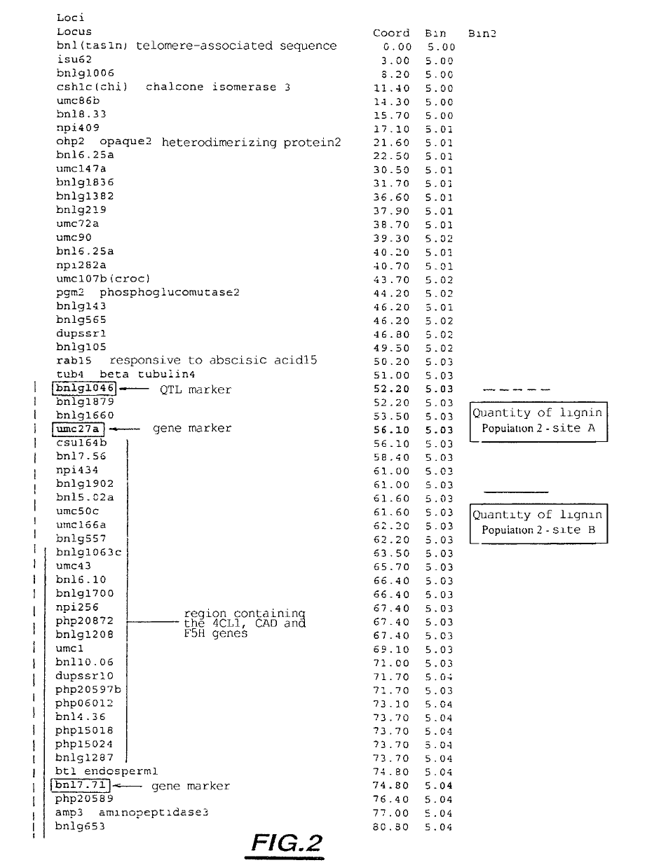

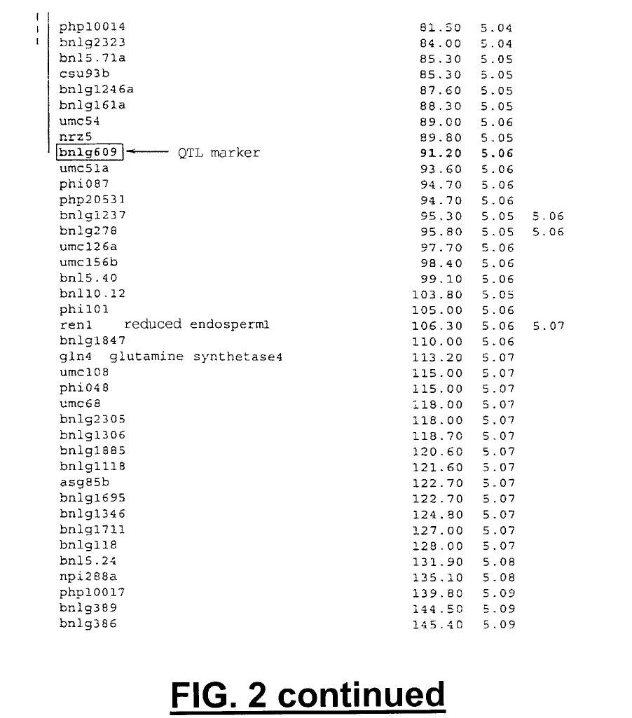

### EXAMPLES

### Example 1

Localization of the Digestibility QTLS:

A. Measurement of Digestibility:

The plant material used is composed of two F2 corn populations (of 220 and 140 individuals respectively) obtained by crossing between flint lines for one and dent lines for the other. The parental lines specific to the same cross were chosen for their difference in wall digestibility and in wall content. The F3 families were evaluated by NIR (near-infrared reflectants) on two culture sites for various digestibility parameters (wall content, lignin content and enzymatic digestibility of total organic material).

B. Biometric Studies:

The linkage between genotype of the marker loci and the quantitative variables of digestibility was established using the method based on an algorithm for searching for QTL by interval mapping (Mapmaker/QTL program version 1.1, Lincoln et al., 1993). A QTL was declared to be significant when the Lod score was greater than 2.5. A confidence interval was defined for each QTL, taking the maximum Lod Score−1.5, for each end.

After mapping the F3 families, QTLs were obtained in particular on chromosomes 1 and 5. The confidence interval (=maximum Lod score−1.5) of the QTL on chromosome 1 is between the markers UMC 67 and UMC 128 and this QTL exhibits, for lignin content, a Lod score of 6.3 for a population and a culture site, and of 2.8 for the other population on the same culture site. The determination coefficients (R²) given by the Mapmaker/QTL program are 20% and 10%, respectively, of the total phenotypic variance. With the same confidence interval, a QTL for enzymatic digestibility of the organic material is shown for a culture site and a population. This QTL has a Lod score of 4.5 and an R² of 13% of the total phenotypic variance.

The QTL obtained on chromosome 5 exhibits a confidence interval included between the markers bnlg 1046 and bnlg 609. This QTL is found for the two culture sites of one of the two populations with a Lod score of 3.1 and 2.8 and an R² of 12% and 10%, respectively, depending on the culture site.

### Example 2

Colocalization of the 4CL2 and CCR Genes:

Two cDNAs, one encoding 4CL2, the other encoding CCR, were mapped based on a segregated population, and a 1.7% recombination distance was observed between these two cDNAs (1 individual recombined out of 60). This region is located on chromosome 1 between the markers UMC 58 and UMC 128.

The cDNAs corresponding to these genes are mapped by hybridization on blots of DNA from a population of segregating individuals, digested with restriction enzymes. The genetic maps of these populations are saturated with at least one marker on average every 4 cM. The program Mapmaker/Exp version 3.0 is used to map the cDNAs relative to the anchoring markers of these genetic maps.

### Example 3

Colocalization of the CAD, 4CL1 and F5H Genes:

The protocol is similar to that followed for colocalizing the 4CL2 and CCR genes. The region to which CAD, 4CL1 and F5H map is located on chromosome 5 between the markers UMC 27a and bnl 7.71.

### Example 4

### Obtaining More Digestible Lines

A. By Marker-Assisted Selection:

Many uses of the selecting markers can be envisioned. Mention may be made of the assessment and management of genetic resources (Song et al., 1988), gene transfer by backcrossing (Young and Tanksley, 1989), construction of novel genotypes (Lefort-Buson et al., 1990), and haplotyping and the search for phenotype-haplotype association for wider use of the information derived from studying QTLs (screening genetic resources).

In general, the genetic markers bordering the chromosomal region, or included in the chromosomal region, characterized according to the invention may thus be used in a marker-assisted selection program in order to predict the genotype at the character trait locus by virtue, firstly, of the linkage between the genetic marker locus and the QTL corn digestibility locus and, secondly, of the colocalization demonstrated between this QTL locus and the genes encoding enzymes involved in lignin biosynthesis. It is therefore also possible to use, as markers, sequences included in these chromosomal regions, comprising all or part of the 4CL1 and 2, CCR, CAD and/or F5H sequences.

In a first step, the effect on digestibility of the allele at the QTL must be assessed in relation to the alleles of the molecular markers of the chromosomal region comprising one of the two QTLs described above.

Thus, a population of corn varieties or of hybrids can be genotyped using a set of markers mapped beforehand on one of the chromosomal regions described above. For each set of markers, each combination of marker alleles is associated with an allele at the QTL and an assessment of the effect of the allele on digestibility may be made by analysis of variance of the digestibility value of the varieties, using the allele at the QTL as factor modality.

The other possibility for assessing the effect of the alleles at the QTL is the approach by crossing: —either by crossing between two lines or varieties; — or by using a series of related or diallele crosses. The principle is, however the same for these two types of cross since the effect of the allele at the QTL is assessed by comparison of the digestibility value of the progeny of the crosses.

In a second step, two varieties (or two progeny-derived individuals), having, respectively, an allele favorable to a good digestibility value for the QTL of chromosome 1 and for the QTL of chromosome 5, are crossed for the purpose of obtaining an individual derived from the progeny of this new cross, which contains these two alleles. Molecular markers belonging to these two chromosomal regions are then used to identify this individual. This individual may then be fixed by successive self pollinations, using the molecular markers to make sure that the two alleles are not lost. Using this individual, or using the parental lines of this individual, the two alleles may also be introgressed into a line or variety exhibiting good agronomic value, and this introgression is followed using molecular markers bordering the region of the QTLs the introgression of which is desired.

B. By Transgenesis:

It is also possible to obtain more digestible plants by the transgenesis pathway, by adjusting the level of expression of the genes encoding enzymes involved in lignin biosynthesis (overexpression of F5H and under-expression of the others for example) or by introducing one or more sequences of genes involved in lignin biosynthesis, these being sequences which correspond to alleles associated with high digestibility values. Expression cassettes are constructed using the sequences localized in the chromosomal regions defined according to the present invention, genetically linked to regulatory sequences of the promoter (strong promoters or promoters specific for highly lignified tissues) and terminator (nos poly A for example) type. These cassettes are then integrated into expression vectors also containing selector markers for the transformation, according to the standard techniques described, for example, in Sambrook et al. (1989). Finally, these vectors are used to transform the plant cells according to the techniques known to those skilled in the art. Mention may be made, in particular, of transformation by particle gun and transformation by agrobacterium, described below.

The use of tissue-specific or organ-specific promoters which allow expression of the transgenes, and therefore improvement of the digestibility, in only a part of the plants may be envisioned. Since it is known that an increase in digestibility linked to a decrease in the quantity of lignin is accompanied, in certain cases, by an increase in sensitivity to beating down (bm3 mutant=mutant of the CAOMT gene), it is advantageous to increase the digestibility by expressing the transgenes throughout the plant except in the stem.

B-1 Particle Gun

The method used is based on the use of a particle gun identical to that described by J. Finer (1992). The target cells are rapidly dividing undifferentiated cells which have conserved the ability to regenerate whole plants. This type of cell makes up the embryogenic callus (termed type II) of corn. These calluses are obtained from immature embryos of the HiII genotype according to the method and on the media described by Armstrong (Maize Handbook; 1994 M. Freeling, V. Walbot Eds; pp. 665-671). 4 h before bombarding, these callus fragments, with a surface area of 10 to 20 mm², were placed, in a proportion of 16 fragments per dish, at the center of a Petri dish containing a culture medium identical to the initiating medium, supplemented with 0.2 M of mannitol+0.2 M of sorbitol. Plasmids carrying the genes to be introduced, in this case 4CL1, 4CL2, CAD, CCR and/or F5H, are purified on a Qiagen^{R} column according to the manufacturer's instructions. They are then precipitated onto particles of tungsten (M10) according to the protocol described by Klein (1987). The particles thus coated are projected onto the target cells using the gun and according to the protocol described by J. Finer (1992). The dishes of calluses thus bombarded are then sealed using Scellofrais^{R} and then cultured in the dark at 27° C. The first subculturing takes place 24 h later, and then every two weeks for 3 months on medium identical to the initiating medium supplemented with a selective agent. 3 months later, or sometimes earlier, calluses are obtained, the growth of which is not inhibited by the selective agent and which are usually and mainly composed of cells resulting from the division of a cell having integrated, into its genetic inheritance, one or more copies of the selection gene. The frequency of production of such calluses is approximately 0.8 callus per dish bombarded.

These calluses are identified, separated, amplified and then cultured so as to regenerate plantlets, modifying the hormonal and osmotic balance of the cells according to the method described by Vain et al. (1989). These plants are then acclimatized in a greenhouse where they may be crossed so as to obtain hybrids or self pollinated.

B-2 Transformation with Agrobacterium

Another transformation technique which can be used in the context of the invention uses Agrobacterium tumefaciens, according to the protocol described by Ishida et al (1996), in particular on immature embryos taken 10 days after pollination. All the media used are referenced in the reference cited. The transformation begins with a coculturing phase in which the immature embryos of the corn plants are brought into contact with Agrobacterium tumefaciens LBA 4404 containing the superbinary vectors, for at least 5 minutes. The superbinary plasmid is the result of homologous recombination between an intermediate vector carrying the T-DNA containing the gene of interest (in this case 4CL1, 4CL2, CAD, CCR and/or F5H) and the Japan Tobacco vector pSB1 (EP 672 752) which contains: the virB and virG genes of the plasmid pTiBo542 present in the supervirulent Agrobacterium tumefaciens strain A281 (ATCC 37349) and a homologous region found in the intermediate vector allowing this homologous recombination. The embryos are then placed on LSAs medium for 3 days in the dark at 25° C. A first selection is carried out on the transformed calluses: the embryogenic calluses are transferred onto LSD5 medium containing phosphinotricine at 5 mg/l and cefotaxim at 250 mg/l (elimination or limitation of the contamination with Agrobacterium tumefaciens). This step is carried out for 2 weeks in the dark and at 25° C. The second selection step is carried out by transferring the embryos which have developed on LSD5 medium, onto LSD10 medium (phosphinotricine at 10 mg/l) in the presence of cefotaxim, for 3 weeks under the same conditions as previously. The third selection step consists in excising the type I calluses (fragments of 1 to 2 mm) and in transferring them onto LSD10 medium, in the presence of cefotaxim, for 3 weeks in the dark at 25° C.

The plantlets are regenerated by excising the type I calluses which have proliferated and transferring them onto LSZ medium in the presence of phosphinotricine at 5 mg/l and cefotaxim for 2 weeks at 22° C. and under continuous light.

The plantlets which have regenerated are transferred onto RM+G2 medium containing 100 mg/l of augmentin for 2 weeks at 22° C. and under continuous illumination for the development step. The plants obtained are then transferred to a phytotron for the purpose of acclimatizing them.

### Example 5

Use of the Population Derived from the Symphony® Hybrid

In a similar manner, the invention may be carried out using a population derived from the Symphony® hybrid as starting plant material.

### BIBLIOGRAPHIC REFERENCES

Allina S M et al, 4-Coumarate:coenzyme A ligase in hybrid poplar. Properties of native enzymes, cDNA cloning, and analysis of recombinant enzymes. Plant Physiol, February 1998, 116(2):743-54.

Aufrere J et al, Predicting organic matter digestibility of forage by two pepsin-cellulase methods. XVI Congrès International des herbages, 1989.

Beavis WD et al: 1991 Quantitative trait loci for plant height in four maize populations and their associations with qualitative genetic loci Then, Appl. Gent. 83:141-145.

Civardi L et al, Molecular cloning and characterisation of two cDNAs encoding enzymes required for secondary cell wall biosynthesis in Maize. In Cellular integration of signalling pathways in plant development. Edited by Schiavo F L, Last R L, Morelli G and Raikhel N V—Springer-Verlag Berlin Heidelberg, 1998.

D de Vienne, 1998, Les marqueurs moléculaires en génétique et biotechnologies végétales, Mieux comprendre [Molecular markers in plant genetics and biotechnology, a better understanding], Editions INRA [INRA publications].

T. Helentjaris, 1987 A linkage map for maize based on RFLPs. Trends Genet 3:217-221.

Hu W J et al, Compartmentalized expression of two structurally and functionally distinct 4-coumarate:CoA ligase genes in aspen (Populus tremuloides). Proc Natl Acad Sci USA, Apr. 28, 1998, 95(9):5407-12

Jung H G et al, Characteristics of plant cell walls affecting intake and digestibility of forages by ruminants. J Anim Sci. September 1995, 73(9):2774-90

Lander and Botstein 1989 Mapping mendelian factors underlying quantitative traits using RFLP linkage maps. Genetics 121:185-199

Lefort-Buson M, De nouvelles perspectives pour l'analyse génétique des caractères quantitatifs. I-A la recherche des loci importants [Novel perspectives for the genetic analysis of quantitative characteristics. I—In search of important loci] Biofutur (1990) 91:30-37

Lincoln E S et al, Constructing genetic linkage map with Mapmaker/EXP version 3.0: a tutorial and reference manual. Whitehead Institute Technical Report, Cambridge, 3rd Edition, January, 1993.

Lincoln ES et al, Mapping genes controlling quantitative traits using Mapmaker/QTL version 1.1: a tutorial and reference manual. Whitehead Institute Technical Report, Cambridge, 2nd Edition, January, 1993.

Lubberstedt T et al, QTL mapping in testcross of European Flint Lines of Maize: II comparison across populations for forage traits. Crop Sci, 1998 38:1278-1289.

Lubberstedt T et al, QTL mapping in testcross of European Flint Lines of Maize: II comparison of different testers for forage quality traits. Crop Sci, 1997 37:1913-1922

Menke K H et al, The estimation of the digestibility and metabolizable energy content of ruminant feedingstuff from the gas production when they are incubated with rumen liquor in vitro. J. Agric Sci (Cambridge), 1979, 93:217-222.

Ronsin T et al, Use of NIRS determination quality in a silage Maize breeding program. The proceedings of the Third International Near Infrared Spectroscopy Conference. Jun. 25-29, 1990 in Brussels—Belgium.

Song K M, Assessment of the degree of restriction fragment length polymorphism in Brassica. Theoretical and Applied Genetics (1988) 75:833-840

Tanksley et al, (1982), Heredity, No. 49, pp. 11-25

Van Soest, Use of the detergents in the analysis of fribrous feeds. J. Assoc. Offic. Agric. Chem. 1963 46:825-835.

Young N D and Tanksley S D, Graphics-based whole genome selection using RFLPs. Current communications in molecular biology—Development and application of molecular markers to problem in plant genetics. (1989): 123-129

1. A chromosomal region identified as being a QTL corn digestibility locus, characterized in that it is delimited by the markers UMC 67 and UMC 128 on corn chromosome 1.

2. A chromosomal region identified as being a QTL corn digestibility locus, characterized in that it is included between the markers UMC 67 and UMC 128 on corn chromosome 1, and in that it comprises the 4CL2 gene encoding the 4-coumarate CoA ligase 2 enzyme and the CCR gene encoding the cynnamoyl CoA reductase enzyme.

3. A chromosomal region, identified as being a QTL corn digestibility locus, characterized in that it is delimited by the markers bnlg 1046 and bnlg 609 on corn chromosome 5.

4. A chromosomal region identified as being a QTL corn digestibility locus, characterized in that it is included between the markers bnlg 1046 and bnlg 609 on corn chromosome 5, and in that it comprises the 4CL1 gene encoding the 4-coumarate CoA ligase 1 enzyme, the CAD gene encoding the cinnamyl alcohol dehydrogenase enzyme and the F5H gene encoding the ferulate 5-hydroxylase enzyme.

5. The use of at least one nucleic acid probe or primer obtained from all or part of the chromosomal region as defined in any one of claims 1 to 4, for identifying, in a corn, the genotype at least partially responsible for high digestibility.

6. The use of at least one nucleic acid probe or primer obtained from all or part of the chromosomal region as defined in any one of claims 1 to 4, for following the introgression of the corn digestibility QTL, in a marker-assisted corn selection program.

7. A method for determining a correlation between the haplotype of the chromosomal regions identified as being a QTL corn digestibility locus and its digestibility value, comprising:
a) haplotyping all or part of the chromosomal region identified as being a QTL digestibility locus, as defined in any one of claims 1 to 4, this haplotyping step being carried out on individuals belonging to the progeny of corns on which the chromosomal regions of one of claims 1 to 4 have been identified as being a QTL corn digestibility locus; and/or
b) haplotyping all or part of the chromosomal region of the QTL digestibility locus as defined in any one of claims 1 to 4, this haplotyping step being carried out on individuals which are not related to said individuals of step (a);
c) determining the digestibility value for all said individuals;
d) establishing a correlation between the digestibility value and the haplotype profiles obtained.

8. A method for the predictive determination of the digestibility value of a corn, comprising:
a) the haplotyping of all or part of the chromosomal region identified as being a QTL digestibility locus, as defined in any one of claims 1 to 4;
b) the predictive determination of the digestibility value of said corn, by means of the correlation established in claim 7.

9. A method for selecting corn with high digestibility, comprising:
haplotyping all or part of the chromosomal region identified as being a QTL digestibility locus, as defined in any one of claims 1 to 4;
selecting, from these individuals, the plants which have a high predictive digestibility value determined using the method of claim 8.

10. A method for selecting corn which has a high degree of digestibility, comprising:
genotyping individuals using nucleic acid probes or primers obtained from all or part of the chromosomal region as defined in any one of claims 1 to 4 or bordering it;
selecting, from these individuals, the plants which comprise a high frequency of favorable alleles associated with digestibility.

11. A method for obtaining genetically transformed corn having high digestibility, said method comprising:
(i) transforming a corn plant cell with at least two nucleotide sequences encoding enzymes which are different from one another, said sequences being chosen from a sequence of an allele favorable to high digestibility encoding CCR, a sequence of an allele favorable to high digestibility encoding 4CL2, a sequence of an allele favorable to high digestibility for 4CL1, a sequence of an allele favorable to high digestibility encoding CAD and a sequence of an allele favorable to high digestibility encoding F5H, said sequences being carried by one or more nucleic acids; and
(ii) regenerating a transgenic plant from the cell thus transformed.
